(19) United States
(12) Patent Application Publication
(10) Pub. No.: US 2025/0197500 A1
(43) Pub. Date: Jun. 19, 2025

(54) METHODS OF TREATING CANCER

(71) Applicant: Coherus BioSciences, Inc., Redwood City, CA (US)

(72) Inventor: Sanjay D. Khare, Palo Alto, CA (US)

(21) Appl. No.: 18/849,692

(22) PCT Filed: Mar. 27, 2023
(86) PCT No.: PCT/US2023/016377

§ 371 Date: Sep. 23, 2024

Related U.S. Application Data
(60) Provisional application No. 63/324,943, filed on Mar. 29, 2022.

(51) Int. Cl. C07K 16/28; A61P 35/00

(52) U.S. Cl. CPC C07K 16/2818; A61P 35/00; C07K 16/2803; C07K 16/2827; C07K 16/2866

(57) ABSTRACT

Provided herein are methods for predicting efficacy of cancer therapeutics in subjects. Also disclosed are methods for treating subjects having a cancer. Provided herein are methods of treating a subject having a cancer that include administering a therapeutically effective amount of an immune checkpoint inhibitor to a subject previously identified or diagnosed as having a cancer and previously identified as having a decreased level of M2 macrophages as compared to a reference level.

### CROSS-REFERENCE TO RELATED APPLICATIONS

This application is a claims the benefit of priority to U.S. Provisional Application No. 63/324,943, filed on Mar. 29, 2022, the contents of which are hereby incorporated by reference.

### SUMMARY

Provided herein are methods of treating a subject having a cancer that include administering a therapeutically effective amount of an immune checkpoint inhibitor to a subject previously identified or diagnosed as having a cancer and previously identified as having a decreased level of M2 macrophages as compared to a reference level.

Also provided herein are methods of selecting a pharmaceutical composition comprising an immune checkpoint inhibitor for a subject previously identified or diagnosed as having a cancer and previously identified as having a decreased level of M2 macrophages as compared to a reference level.

Also provided herein are methods of selecting a subject previously identified or diagnosed as having a cancer and previously identified as having a decreased level of M2 macrophages for treatment with a pharmaceutical composition comprising an immune checkpoint inhibitor.

Also provided herein are methods of predicting efficacy of treatment with a pharmaceutical composition comprising an immune checkpoint inhibitor in a subject in need thereof that include: determining that a subject diagnosed or identified as having a cancer and identified as having a decreased level of M2 macrophages as compared to a reference level has an increased likelihood of having a positive therapeutic response to treatment with the pharmaceutical composition; or determining that a subject diagnosed or identified as having a cancer and identified as having an elevated level of M2 macrophages as compared to a reference level has a decreased likelihood of having a positive therapeutic response to treatment with the pharmaceutical composition.

In some embodiments of any of the methods described herein, the immune checkpoint inhibitor is an anti-PD-1 antibody or an antigen-binding fragment thereof. In some embodiments of any of the methods described herein, the immune checkpoint inhibitor is an anti-PD-L1 antibody or an antigen-binding fragment thereof.

In some embodiments of any of the methods described herein, the method further comprises: determining a level of M2 macrophages in a sample obtained from the subject; and identifying the subject as having an increased (or elevated) level of M2 macrophages as compared to the reference level.

In some embodiments of any of the methods described herein, the method further comprises: determining a level of M2 macrophages in a sample obtained from the subject; and identifying the subject as having a decreased level of M2 macrophages as compared to the reference level.

Also provided herein are methods of treating a subject having a cancer by administering a therapeutically effective amount of an anti-ILT4 binding agent to a subject previously identified or diagnosed as having a cancer and previously identified as having an elevated level of M2 macrophages as compared to a reference level.

Also provided herein are methods of selecting a pharmaceutical composition comprising an anti-ILT4 binding agent for a subject previously identified or diagnosed as having a cancer and previously identified as having an elevated level of M2 macrophages as compared to a reference level.

Also provided herein are methods of selecting a subject previously identified or diagnosed as having a cancer and previously identified as having an elevated level of M2 macrophages for treatment with a pharmaceutical composition comprising an anti-ILT4 binding agent.

In some embodiments of any of the methods described herein, the anti-ILT4 binding agent is an anti-ILT4 antibody or an antigen-binding fragment thereof (e.g., a blocking antibody that blocks ILT4/LILRB2 interaction with HLA-G molecule).

Some embodiments of any of the methods described herein further include administering an immune checkpoint inhibitor to the subject. In some embodiments of any of the methods described herein, the immune checkpoint inhibitor is an anti-PD-1 antibody or an antigen-binding fragment thereof. In some embodiments of any of the methods described herein, the immune checkpoint inhibitor is an anti-PD-L1 antibody or an antigen-binding fragment thereof.

Some embodiments of any of the methods described herein further include: determining a level of M2 macrophages in a sample obtained from the subject; and identifying the subject as having an elevated level of M2 macrophages as compared to the reference level.

In some embodiments of any of the methods described herein, the M2 macrophages have an elevated level of ILT4 expression as compared to a reference level.

In some embodiments of any of the methods described herein, the reference level of M2 macrophages is a level of M2 macrophages in a population of healthy subjects.

In some embodiments of any of the methods described herein, the reference level of M2 macrophages is a 75% percentile of the median level of the number of M2 macrophages in a healthy patient population. In some embodiments of any of the methods described herein, the reference level of M2 macrophages is a 80% percentile of the median level of M2 macrophages in a healthy patient population. In some embodiments of any of the methods described herein, the reference level of M2 macrophages is a 85% percentile of the median level of M2 macrophages in a healthy patient population. In some embodiments of any of the methods described herein, the reference level of M2 macrophages is a 90% percentile of the median level of M2 macrophages in a healthy patient population. In some embodiments of any of the methods described herein, the reference level of M2 macrophages is a 95% percentile of the median level of M2 macrophages in a healthy patient population.

In some embodiments of any of the methods described herein, the cancer is a solid tumor. In certain embodiments of any of the methods described herein, the solid tumor is non-small cell lung cancer (NSCLC), hepatocellular carcinoma (HCC), small-cell lung cancer (SCLC), head and neck cancer, or esophageal cancer.

In some embodiments of any of the methods described herein, the cancer is a hematological cancer.

Also provided herein are methods of treating a subject having a cancer that include administering a therapeutically effective amount of an anti-CCR8 binding agent to a subject previously identified or diagnosed as having a cancer and previously identified as having an elevated level of Treg cells and an elevated level of NK cells as compared to reference levels.

Also provided herein are methods of selecting a pharmaceutical composition comprising an anti-CCR8 binding agent for a subject previously identified or diagnosed as having a cancer and previously identified as having an elevated level of Treg cells and an elevated level of NK cells as compared to reference levels.

Also provided herein are methods of selecting a subject previously identified or diagnosed as having a cancer and previously identified as having an elevated level of Treg cells and an elevated level of NK cells as compared to reference levels for treatment with a pharmaceutical composition comprising an anti-CCR8 binding agent.

Also provided herein are methods of predicting efficacy of treatment with a pharmaceutical composition comprising an anti-CCR8 binding agent in a subject in need thereof that include: determining that a subject diagnosed or identified as having a cancer and identified as having an elevated level of Treg cells and an elevated level of NK cells as compared to reference levels has an increased likelihood of having a positive therapeutic response to treatment with the pharmaceutical composition; or determining that a subject diagnosed or identified as having a cancer and identified an elevated level of Treg cells and a decreased level of NK cells as compared to reference levels has a decreased likelihood of having a positive therapeutic response to treatment with the pharmaceutical composition.

In some embodiments of any of the methods described herein, the anti-CCR8 binding agent is an anti-CCR8 antibody or antigen-binding fragment thereof (e.g., a depleting antibody or an antigen-binding fragment thereof with an ability to block ligand binding to CCR8).

In some embodiments of any of the methods described herein, the method further comprises administering an immune checkpoint inhibitor to the subject. In some embodiments of any of the methods described herein, the immune checkpoint inhibitor is an anti-PD-1 antibody or an antigen-binding fragment thereof. In some embodiments of any of the methods described herein, the immune checkpoint inhibitor is an anti-PD-L1 antibody or an antigen-binding fragment thereof.

In some embodiments of any of the methods described herein, the method further comprises: determining a level of Treg cells and NK cells in a sample obtained from the subject; and identifying the subject as having an elevated level of T reg cells and NK cells as compared to the reference levels.

In some embodiments of any of the methods described herein, the reference level of Treg cells is a level of Treg cells in a population of healthy subjects.

In some embodiments of any of the methods described herein, the reference level of Treg cells is a 75% percentile of the median level of the number of Treg cells in a healthy patient population. In some embodiments of any of the methods described herein, the reference level of Treg cells is a 80% percentile of the median level of Treg cells in a healthy patient population. In some embodiments of any of the methods described herein, the reference level of Treg cells is a 85% percentile of the median level of Treg cells in a healthy patient population. In some embodiments of any of the methods described herein, the reference level of Treg cells is a 90% percentile of the median level of Treg cells in a healthy patient population. In some embodiments of any of the methods described herein, the reference level of Treg cells is a 95% percentile of the median level of Treg cells in a healthy patient population.

In some embodiments of any of the methods described herein, the reference level of NK cells is a level of NK cells in a population of healthy subjects.

In some embodiments of any of the methods described herein, the reference level of NK cells is a 75% percentile of the median level of the number of NK cells in a healthy patient population. In some embodiments of any of the methods described herein, the reference level of NK cells is a 80% percentile of the median level of NK cells in a healthy patient population. In some embodiments of any of the methods described herein, the reference level of NK cells is a 85% percentile of the median level of NK cells in a healthy patient population. In some embodiments of any of the methods described herein, the reference level of NK cells is a 90% percentile of the median level of NK cells in a healthy patient population. In some embodiments of any of the methods described herein, the reference level of NK cells is a 95% percentile of the median level of NK cells in a healthy patient population.

In some embodiments of any of the methods described herein, the cancer is a solid tumor. In certain embodiments of any of the methods described herein, the solid tumor is non-small cell lung cancer (NSCLC), hepatocellular carcinoma (HCC), small-cell lung cancer (SCLC), head and neck cancer, or esophageal cancer.

In some embodiments of any of the methods described herein, the cancer is a hematological cancer.

Also provided herein are methods of treating a subject having a cancer that include administering a therapeutically effective amount of an anti-CCR8 binding agent and a therapeutically effective amount of anti-ILT4 binding agent to a subject previously identified or diagnosed as having a cancer and previously identified as having an elevated level of Treg cells, an elevated level of NK cells, and an elevated number of M2 macrophages as compared to reference levels.

Also provided herein are methods of selecting an anti-CCR8 binding agent and an anti-ILT4 binding agent for a subject previously identified or diagnosed as having a cancer and previously identified as having an elevated level of Treg cells, an elevated level of NK cells, and an elevated level of NK cells as compared to reference levels.

Also provided herein are methods of selecting a subject previously identified or diagnosed as having a cancer and previously identified as having an elevated level of Treg cells, an elevated level of NK cells, and an elevated level of M2 macrophages as compared to reference levels for treatment with an anti-CCR8 binding agent and an anti-ILT4 binding agent.

Also provided herein are methods of predicting efficacy of treatment with an anti-CCR8 binding agent and an anti-ILT4 binding agent in a subject in need thereof that include: determining that a subject diagnosed or identified as having a cancer and identified as having an elevated level of Treg cells, an elevated level of NK cells, and an elevated level of M2 macrophages as compared to reference levels has an increased likelihood of having a positive therapeutic response to treatment with the pharmaceutical composition; or determining that a subject diagnosed or identified as having a cancer and not identified as having an elevated level of Treg cells, an elevated level of NK cells, and an elevated level of M2 macrophages as compared to reference levels has a decreased likelihood of having a positive therapeutic response to treatment with an anti-CCR8 binding agent and an anti-ITL4 binding agent.

In some embodiments of any of the methods described herein, the anti-CCR8 binding agent is an anti-CCR8 antibody or antigen-binding fragment thereof (e.g., a depleting antibody or an antigen-binding fragment thereof with an ability to block ligand binding to CCR8).

In some embodiments of any of the methods described herein, the anti-ILT4 binding agent is an anti-ILT4 antibody or an antigen-binding fragment thereof (e.g., a blocking antibody that blocks ILT4/LILRB2 interaction with HLA-G molecule).

In some embodiments of any of the methods described herein, the method further comprises administering an immune checkpoint inhibitor to the subject. In some embodiments of any of the methods described herein, the immune checkpoint inhibitor is an anti-PD-1 antibody or an antigen-binding fragment thereof. In some embodiments of any of the methods described herein, the immune checkpoint inhibitor is an anti-PD-L1 antibody or an antigen-binding fragment thereof.

In some embodiments of any of the methods described herein, the method further comprises: determining a level of Treg cells, NK cells, and M2 macrophages in a sample obtained from the subject; and identifying the subject as having an elevated level of T reg cells, an elevated level of NK cells, and an elevated level of M2 macrophages as compared to the reference levels.

In some embodiments of any of the methods described herein, the reference level of Treg cells is a level of Treg cells in a population of healthy subjects.

In some embodiments of any of the methods described herein, the reference level of Treg cells is a 75% percentile of the median level of the number of Treg cells in a healthy patient population. In some embodiments of any of the methods described herein, the reference level of Treg cells is a 80% percentile of the median level of Treg cells in a healthy patient population. In some embodiments of any of the methods described herein, the reference level of Treg cells is a 85% percentile of the median level of Treg cells in a healthy patient population. In some embodiments of any of the methods described herein, the reference level of Treg cells is a 90% percentile of the median level of Treg cells in a healthy patient population. In some embodiments of any of the methods described herein, the reference level of Treg cells is a 95% percentile of the median level of Treg cells in a healthy patient population.

In some embodiments of any of the methods described herein, the reference level of NK cells is a level of NK cells in a population of healthy subjects.

In some embodiments of any of the methods described herein, the reference level of NK cells is a 75% percentile of the median level of the number of NK cells in a healthy patient population. In some embodiments of any of the methods described herein, the reference level of NK cells is a 80% percentile of the median level of NK cells in a healthy patient population. In some embodiments of any of the methods described herein, the reference level of NK cells is a 85% percentile of the median level of NK cells in a healthy patient population. In some embodiments of any of the methods described herein, the reference level of NK cells is a 90% percentile of the median level of NK cells in a healthy patient population. In some embodiments of any of the methods described herein, the reference level of NK cells is a 95% percentile of the median level of NK cells in a healthy patient population.

In some embodiments of any of the methods described herein, the reference level of M2 macrophages is a level of M2 macrophages in a population of healthy subjects.

In some embodiments of any of the methods described herein, the reference level of M2 macrophages is a 75% percentile of the median level of the number of M2 macrophages in a healthy patient population. In some embodiments of any of the methods described herein, the reference level of M2 macrophages is a 80% percentile of the median level of M2 macrophages in a healthy patient population. In some embodiments of any of the methods described herein, the reference level of M2 macrophages is a 85% percentile of the median level of M2 macrophages in a healthy patient population. In some embodiments of any of the methods described herein, the reference level of M2 macrophages is a 90% percentile of the median level of M2 macrophages in a healthy patient population. In some embodiments of any of the methods described herein, the reference level of M2 macrophages is a 95% percentile of the median level of M2 macrophages in a healthy patient population.

In some embodiments of any of the methods described herein, the cancer is a solid tumor. In certain embodiments of any of the methods described herein, the solid tumor is non-small cell lung cancer, hepatocellular carcinoma, small-cell lung cancer, head and neck cancer, or esophageal cancer.

In some embodiments of any of the methods described herein, the cancer is a hematological cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B provide graphs showing association of high level of immune suppressive M2 macrophages with limited PD(L)-1 response in small cell lung cancer (SCLC) patients. FIG. 1A is a graph showing re-analysis of survival probability data from the patients. FIG. 1B is a graph showing re-analysis of heat map data from the patients.

FIGS. 2A-2B provide graphs showing ILT4 expression is higher and immune-T cells are lower in SCLC patients that have higher level of M2 macrophages. FIG. 2A is a graph showing ILT4 expression in M2 vs. non-M2 samples. FIG. 2B is a graph showing CD8 T cells, CD4-Naïve T cells, CD4-Memory-Resting T cells, and CD4-Memory-Activated T cells in M2-enriched samples. **** p<0.00001

FIG. 3 is a graph showing high expression of immune suppressive M2 macrophages in ˜30% of non-small cell lung cancer (NSCLC) patients.

FIG. 4 is a schematic showing repolarization of M2 (suppressive) macrophages to M1 (inflammatory) macrophages by anti-ILT4 antibody.

FIG. 5 is a graph showing re-analysis of heat map data in NSCLC patients. About 30% of the patients had high Treg signatures with Population 1 having High Tregs and High NK cells (Treg^{high}NK^{high}), and Population 2 having High Tregs and Low NK cells (Treg^{high}NK^{low}).

FIG. 6 is a schematic showing Treg cells interfering with anti PD(L)1 efficacy (Left panel) and effective tumor killing with CCR8 antibody in the presence of NK cells (Right panel).

FIG. 7 is a graph showing patient populations that will benefit most from new combination therapies comprising ILT4 and CCR8 in combination with toripalimab. The graph shows percentage of patients in each of two identified populations (i.e., “Treg Hi, Effector Hi” population and “M2 Enriched” population) that are likely to benefit from the combination therapy. Data is shown for hepatocellular carcinoma (HCC), NSCLC adenocarcinoma, SCLC, head and neck cancers, and esophageal cancer. SCLC M1 and M2 values are based on ADAPTS: LM22 profile. Treg pattern is derived from GSVA using ranked LM22 profile.

### DETAILED DESCRIPTION

The present disclosure provides methods for predicting efficacy of cancer therapeutics in subjects (e.g., cancer patients). For example, the present disclosure provides methods for predicting efficacy of immune checkpoint inhibitors (ICIs), anti-CCR8 binding agents (e.g., an anti-CCR8 antibody, such as a depleting antibody), and/or anti-ILT4 binding agents (e.g., an anti-ILT4 antibody, such as a blocking antibody) in cancer patients. Also disclosed are methods for treating subjects (e.g., patients) having one or more cancers with cancer therapeutics. For example, the present disclosure provides methods for treating subjects having one or more cancers with ICIs, anti-CCR8 binding agents, anti-ILT4 binding agents, and/or any combinations thereof.

### Predicting Efficacy of Cancer Therapeutics

Provided herein are methods of predicting efficacy of treatment with a pharmaceutical composition comprising an immune checkpoint inhibitor in a subject in need thereof (e.g., a subject having any of the cancers described herein) that include: determining that a subject diagnosed or identified as having a cancer and identified as having a decreased level of M2 macrophages as compared to a reference level has an increased likelihood of having a positive therapeutic response to treatment with the pharmaceutical composition; or determining that a subject diagnosed or identified as having a cancer and identified as having an elevated level of M2 macrophages as compared to a reference level has a decreased likelihood of having a positive therapeutic response to treatment with the pharmaceutical composition.

Also provided herein are methods of predicting efficacy of treatment with a pharmaceutical composition comprising an anti-CCR8 binding agent in a subject in need thereof (e.g., a subject having any of the exemplary cancers described herein) that include: determining that a subject diagnosed or identified as having a cancer and identified as having an elevated level of Treg cells and an elevated level of NK cells as compared to reference levels has an increased likelihood of having a positive therapeutic response to treatment with the pharmaceutical composition; or determining that a subject diagnosed or identified as having a cancer and identified an elevated level of Treg cells and a decreased level of NK cells as compared to reference levels has a decreased likelihood of having a positive therapeutic response to treatment with the pharmaceutical composition.

Also provided herein are methods of predicting efficacy of treatment with an anti-CCR8 binding agent and an anti-ILT4 binding agent in a subject in need thereof (e.g., a subject having any of the exemplary cancers described herein) that include: determining that a subject diagnosed or identified as having a cancer and identified as having an elevated level of Treg cells, an elevated level of NK cells, and an elevated level of M2 macrophages as compared to reference levels has an increased likelihood of having a positive therapeutic response to treatment with the pharmaceutical composition; or determining that a subject diagnosed or identified as having a cancer and not identified as having an elevated level of Treg cells, an elevated level of NK cells, and an elevated level of M2 macrophages as compared to reference levels has a decreased likelihood of having a positive therapeutic response to treatment with an anti-CCR8 binding agent and an anti-ITL4 binding agent.

### M2 Macrophages

In contrast to the classically activated macrophages (also known as M1 macrophages), M2 macrophages are alternatively activated. M2 macrophages are immune suppressive in nature. Unlike the inflammatory M1 macrophages, M2 macrophages are prone to promote angiogenesis and neovascularization, as well as stromal activation and remolding, thereby impacting cancer progression positively and patient′ prognosis negatively. M1 macrophages are characterized by the expression of HLA-DR and CD197, whereas M2 has high expression of CD163, CD209, CD206, CCL2, etc. M2 macrophages are described in further detail in the art. See, e.g., Liu et al., Cancer Cell Int, 21:389, 2021; Tiainen et al., Histopathology, 66:873-83, 2015.

As used herein, “reference level” of a parameter may refer to the level of that parameter in a healthy patient (e.g., a patient not having an autoimmune disorder or inflammatory disorder or an allergy) or level of that parameter in a population of healthy subjects. For example, “reference” level of M2 macrophages may refer to the level of M2 macrophages in a healthy patient (e.g., a patient not having an autoimmune disorder or inflammatory disorder or an allergy) or level of M2 macrophages in a population of healthy subjects. In certain instances, the reference level of M2 macrophages is a 55% percentile, a 60% percentile, a 65% percentile, a 70% percentile, a 75% percentile, a 80% percentile, a 85% percentile, a 90% percentile, or a 95% percentile of the median level of the number of M2 macrophages in a healthy patient population.

### Treg Cells

Regulatory T cells (Tregs or Treg cells), as an important mechanism for regulating homeostasis of the immune system and the immune tolerance of the body, play crucial roles in the regulation of tumor immunity. Treg cells can inhibit the activation and differentiation of CD4⁺ helper T cells and CD8⁺ cytotoxic T cells to induce reactivity against autologous and tumor-expressed antigens. In the tumor microenvironment (TME), Treg cells can be induced and differentiated by traditional T cells, which have a strong immunosuppressive function, inhibit antitumor immunity, and promote the occurrence and development of tumors. Treg cells can also suppress the function of immune effector cells through a variety of mechanisms and are key factors in tumor immune escape. Forkhead/winged helix transcription factor (Foxp3) is specifically expressed in Treg cells, and CD4⁺CD25⁺Foxp3⁺ is currently considered to be a classical combined marker of Treg cells. In fact, in addition to its ability to label Treg cells, Foxp3 dominantly controls Treg cell function, and only its continuous expression guarantees the maintenance of full Treg cell suppressive capacity. Treg cells are described in further detail in the art. See, e.g., Li et al., Mol Cancer, 19:116, 2020; Nishikawa and Sakaguchi, Curr Opin Immunol, 27:1-7, 2014.

As used herein, a “reference level” of Treg cells may refer to the level of Treg cells in a healthy patient (e.g., a patient not having an autoimmune disorder or inflammatory disorder or an allergy) or level of Treg cells in a population of healthy subjects. In certain instances, the reference level of Treg cells is a 55% percentile, a 60% percentile, a 65% percentile, a 70% percentile, a 75% percentile, a 80% percentile, a 85% percentile, a 90% percentile, or a 95% percentile of the median level of the number of Treg cells in a healthy patient population.

### NK Cells

Natural killer (NK) cells are lymphocytes with innate ability to lyse tumor cells without the need for prior sensitization. NK cells can trigger target cell death by releasing cytotoxic granules containing granzymes and perforin and through death receptor-mediated pathways (e.g., FasL/Fas). NK cells also play immunomodulatory functions by secreting chemokines and cytokines, such as RANTES and IFN-γ. In humans, NK cells are traditionally identified by the absence of CD3 and the presence of CD56 on their surface as characterized by flow cytometry. NK cells are described in further detail in the art. See, e.g., Liu et al., J Hematol Oncol, 14:7, 2021; Freud et al., Immunity, 47 (5): 820-833, 2017; Spits et al., Nat Rev Immunol, 13 (2): 145-9, 2013. NK cells also have Fc receptors important for the binding of the Fc region of the therapeutic antibody, leading to an activation and degranulation of NK cells for an effective target cell killing.

As used herein, a “reference level” of NK cells may refer to the level of NK cells in a healthy patient (e.g., a patient not having an autoimmune disorder or inflammatory disorder or an allergy) or level of NK cells in a population of healthy subjects. In certain instances, the reference level of NK cells is a 55% percentile, a 60% percentile, a 65% percentile, a 70% percentile, a 75% percentile, a 80% percentile, a 85% percentile, a 90% percentile, or a 95% percentile of the median level of the number of NK cells in a healthy patient population.

### Immune Checkpoint Inhibitors

ICIs are a novel class of immunotherapy drugs that have improved the treatment of a broad spectrum of cancers as metastatic melanoma, non-small lung cancer or renal cell carcinoma. ICIs may comprise humanized and human monoclonal antibodies that target inhibitory receptors (e.g., CTLA-4, PD-1, LAG-3, TIM-3) and ligands (e.g., PD-L1) expressed on T lymphocytes, antigen presenting cells and tumor cells and elicit an anti-tumor response by stimulating immune system. For example, an ICI described herein can be an anti-PD-1 antibody or an antigen-binding fragment thereof, an anti-PD-L1 antibody or an antigen-binding fragment thereof, and/or an anti-CTLA-4 antibody or an antigen-binding fragment thereof.

An overview of different ICIs, including the corresponding immune checkpoints, targeted indications (e.g., type of tumor(s) targeted by those ICIs), and clinical trials (if any), is provided in Table 1. Further details on ICIs can be found in the art. See, e.g., Franzin et al., Front Immunol, 11:574271, 2020; Johnson et al., Nat Rev Clin Oncol, 19:254-267, 2022.

TABLE 1
Overview of principal ICIs, targeted tumor and clinical trials

| Immune | | | | |
|---|---|---|---|---|
| checkpoint | | Year of | FDA-approved | |
| inhibited | Drugs | approval | indications | Clinical Trial |
| CTLA-4 | Ipilimumab | 2011 | Metastatic melanoma, | Non-small cell lung |
| | | | Renal cell carcinoma, | carcinoma |
| | | | Colorectal cancer | NCT03469960, |
| | | | | NCT03351361, |
| | | | | NCT02785952, |
| | | | | NCT03302234 |
| | | | | Mesothelioma |
| | | | | NCT02899299 |
| | | | | Gastric cancer |
| | | | | NCT02872116 |
| | | | | Squamous cell lung |
| | | | | carcinoma |
| | | | | NCT02785952 |
| PD-1 | Nivolumab | 2015 | Metastatic melanoma, | Mesothelioma |
| | | | Colorectal cancer, | NCT03063450 |
| | | | Classical Hodgkin's | Non-Hodgkin |
| | | | lymphoma, | lymphoma |
| | | | Renal cell carcinoma, | NCT03366272 |
| | | | Non-small cell lung | Metastatic clear cell |
| | | | carcinoma, | renal carcinoma |
| | | | Head and neck | NCT01668784 |
| | | | squamous cell | Head and neck cancer |
| | | | carcinoma | NCT02741570, |
| | | | (HNSCC), | NCT03342352 |
| | | | Metastatic urothelial | Lung cancer |
| | | | carcinoma, | NCT03348904 |
| | | | Hepatocellular | |
| | | | carcinoma (HCC), | |
| | | | Colorectal cancer with | |
| | | | MSI-H | |
| | Pembrolizumab | 2015 | Metastatic melanoma, | Small cell lung cancer |
| | | | metastatic NSCLC, | NCT03066778 |
| | | | classical Hodgkin's | Renal cell carcinoma |
| | | | lymphoma, | NCT03142334, |
| | | | primary mediastinal | NCT02853331 |
| | | | B-cell lymphoma | Gastric |
| | | | (PMBCL), | adenocarcinoma |
| | | | Head and neck | NCT02370498 |
| | | | squamous cell | |
| | | | carcinoma | |
| | | | (HNSCC), | |
| | | | gastric cancer, | Urothelial carcinoma |
| | | | solid tumors with | NCT02853305, |
| | | | MSI-H and MMR | NCT03244384, |
| | | | aberrations, | NCT02256436, |
| | | | metastatic urothelial | NCT03374488, |
| | | | carcinoma, | Colorectal cancer |
| | | | Merkel cell | NCT02563002 |
| | | | carcinoma, | Pleural mesothelioma |
| | | | renal cell carcinoma, | NCT02991482 |
| | | | Cervical cancer, | Esophageal neoplasms |
| | | | Hepatocellular | NCT03189719, |
| | | | carcinoma | NCT02564263 |
| | | | | Multiple myeloma |
| | | | | NCT02579863, |
| | | | | NCT02576977 |
| | | | | Hodgkin lymphoma |
| | | | | NCT02684292 |
| | | | | Hepatocellular |
| | | | | carcinoma |
| | | | | NCT02702401, |
| | | | | NCT03062358 |
| | Cemiplimab | 2018 | Metastatic cutaneous | Cutaneous squamous |
| | | | squamous cell | cell carcinoma |
| | | | carcinoma | NCT04154943 |
| PD-L1 | Atezolizumab | 2016 | Metastatic urothelial | Renal cell cancer |
| | | | carcinoma, | NCT02684006 |
| | | | Metastatic Non-small | Gastric and |
| | | | cell lung carcinoma, | gastroesophageal |
| | | | Metastatic Small cell | junction cancer |
| | | | lung carcinoma, | NCT02625623, |
| | | | Metastatic triple | NCT02625610 |
| | | | negative breast cancer | Ovarian cancer, |
| | | | | fallopian tube cancer |
| | | | | NCT03038100, |
| | | | | NCT02839707, |
| | | | | NCT02891824 |
| | Avelumab | 2017 | Merkel cell | Non-small cell lung |
| | | | carcinoma, | carcinoma |
| | | | Metastatic urothelial | NCT02576574, |
| | | | carcinoma | NCT02395172 |
| | | | | Urothelial cancer |
| | | | | NCT02603432 |
| | | | | Diffuse large B-cell |
| | | | | lymphoma |
| | | | | NCT02951156 |
| | Durvalumab | 2018 | Metastatic urothelial | Non-small cell lung |
| | | | carcinoma, | carcinoma |
| | | | Unresectable stage III | NCT02273375, |
| | | | Non-small cell lung | NCT02542293, |
| | | | carcinoma | NCT03164616, |
| | | | | NCT02125461 |
| | | | | Squamous cell lung |
| | | | | carcinoma |
| | | | | NCT02154490, |
| | | | | NCT02551159 |
| | | | | Urothelial cancer |
| | | | | NCT02516241 |
| | | | | Advanced solid |
| | | | | malignancies |
| | | | | NCT03084471 |
| Combination | Ipilimumab | 2016 | Metastatic melanoma | Non-small cell lung |
| of CTLA-4 | plus nivolumab | 2018 | Metastatic renal cell | cancer |
| and PD-1 | | | carcinoma | NCT02659059 |
| | | 2018 | Colorectal cancer with | Metastatic renal cell |
| | | | MSI-H | carcinoma |
| | | 2020 | Hepatocellular | NCT0223174 |
| | | | carcinoma (HCC) | Colorectal cancer with |
| | | | | MSI-H |
| | | | | NCT02060188 |
| | | | | Hepatocellular |
| | | | | carcinoma (HCC) |
| | | | | NCT01658878 |

### Anti-CCR8 Binding Agent

An anti-CCR8 binding agent can be an agent that specifically binds to Chemokine (C-C motif) receptor 8 (CCR8). For example, an anti-CCR8 binding agent can be an anti-CCR8 antibody or a fragment thereof. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications (including, but not limited to the addition of one or more detectable labels). In some embodiments, an anti-CCR8 binding agent (e.g., an anti-CCR8 antibody) can be a depleting antibody. In certain embodiments, the depleting antibody may have the ability to block a ligand from binding to CCR8. In certain instances, non-fucosylated anti-CCR8 antibodies can be used, which have increased antibody-dependent cellular cytotoxicity (ADCC) activity and are therefore good depleters of CCR8-expressing Tregs.

In some instances, BMS-986340 can be used in the methods of the present disclosure as an anti-CCR8 binding agent (e.g., an anti-CCR8 antibody). BMS-986340 is a newly developed anti-CCR8 mAb which reduces sizeable CCR8+ Treg in a human tumor explant. Other anti-CCR8 antibodies for use in the methods of the present disclosure are described in the art. See, e.g., Campbell et al., Cancer Res, 81 (11): 2983-2994, 2021.

### Anti-ILT4 Binding Agent

An anti-ILT4 binding agent can be an agent that specifically binds to inhibitory immune checkpoint receptor immunoglobulin-like transcript 4 (ILT4; leukocyte immunoglobulin-like receptor subfamily B member 2; LILRB2; lymphocyte immunoglobulin-like receptor 2; LIR2; monocyte/macrophage immunoglobulin-like receptor 10; MIR-10; CD85d). For example, an anti-ILT4 binding agent can be an anti-ILT4 antibody or a fragment thereof. In some embodiments, an anti-ILT4 binding agent (e.g., an anti-ILT4 antibody) can be a blocking antibody. In certain embodiments, the blocking antibody can inhibit interaction of ILT4 (LILRB2) with its ligands, including HLA-G.

In some instances, MK-4830 can be used in the methods of the present disclosure as an anti-ILT4 binding agent (e.g., an anti-ILT4 antibody). MK-4830 is a human monoclonal antibody directed against ILT4 with potential immunomodulating and antineoplastic activities. Other anti-ILT4 antibodies for use in the methods of the present disclosure are described in the art. See, e.g., Chen et al., Theranostics 11 (7): 3392-3416, 2021.

### Methods of Treatment

Provided herein are methods of treating a subject having a cancer (e.g., any of the exemplary cancers described herein) that include administering a therapeutically effective amount of an immune checkpoint inhibitor to a subject previously identified or diagnosed as having a cancer (e.g., any of the exemplary cancers described herein) and previously identified as having a increased level of M2 macrophages as compared to a reference level (e.g., any of the exemplary reference levels described herein).

Also provided herein are methods of treating a subject having a cancer (e.g., any of the exemplary cancers described herein) that include: administering a therapeutically effective amount of an anti-ILT4 binding agent to a subject previously identified or diagnosed as having a cancer (e.g., any of the exemplary cancers described herein) and previously identified as having an elevated level of M2 macrophages as compared to a reference level (e.g., any of the exemplary reference levels described herein).

Also provided herein are methods of treating a subject having a cancer (e.g., any of the exemplary cancers described herein) that include: administering a therapeutically effective amount of an anti-CCR8 binding agent to a subject previously identified or diagnosed as having a cancer (e.g., any of the exemplary cancers described herein) and previously identified as having an elevated level of Treg cells and an elevated level of NK cells as compared to reference levels (e.g., any of the exemplary reference levels described herein).

Also provided herein are methods of treating a subject having a cancer (e.g., any of the exemplary cancers described herein) that include: administering a therapeutically effective amount of an anti-CCR8 binding agent and a therapeutically effective amount of anti-ILT4 binding agent to a subject previously identified or diagnosed as having a cancer and previously identified as having an elevated level of Treg cells, an elevated level of NK cells, and an elevated number of M2 macrophages as compared to reference levels (e.g., any of the exemplary reference levels described herein).

As used herein, “treating” or “treatment” refers to alleviating, ameliorating, improving, relieving, delaying onset of, inhibiting progression of, reducing severity of, and/or reducing incidence of one or more symptoms or features of a particular disease, disorder, and/or condition. For example, “treating” cancer may refer to extending survival of a subject diagnosed or identified as having a cancer. Additionally or in the alternative, “treating” cancer may refer to inhibiting growth and/or spread of a cancer or tumor.

As used herein, the term “subject” refers to any organism to which a method of the present disclosure may be applied, e.g., for experimental and/or therapeutic purposes. Typical subjects include animals, such as mammals (e.g., mice, rats, rabbits, dogs, pigs, non-human primates, and humans). In some instances, a “subject” can refer to a cancer patient, such as a one who is diagnosed and/or identified as having a cancer.

Cancers for which the present treatment methods are useful may include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung (including small-cell lung cancer, non-small cell lung cancer, adenocarcinoma of the lung, and squamous carcinoma of the lung), bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

In certain embodiments, the cancer treated in accordance with the methods described herein includes but is not limited to, prostate cancer, breast cancer, lung cancer, colorectal cancer, melanoma, bronchial cancer, bladder cancer, brain or central nervous system cancer, peripheral nervous system cancer, uterine or endometrial cancer, cancer of the oral cavity or pharynx, non-Hodgkin's lymphoma, thyroid cancer, kidney cancer, biliary tract cancer, small bowel or appendix cancer, salivary gland cancer, thyroid gland cancer, adrenal gland cancer, squamous cell cancer, mesothelioma, osteocarcinoma, thyoma/thymic carcinoma, glioblastoma, myelodysplastic syndrome, soft tissue sarcoma, DIPG, adenocarcinoma, osteosarcoma, chondrosarcoma, leukemia, or pancreatic cancer. In some embodiments, the cancer treated in accordance with the methods described herein includes a carcinoma (e.g., an adenocarcinoma), lymphoma, blastoma, melanoma, sarcoma or leukemia. In certain embodiments, the cancer treated in accordance with the methods described herein includes squamous cell cancer, small-cell lung cancer, non-small cell lung cancer, gastrointestinal cancer, Hodgkin's lymphoma, non-Hodgkin's lymphoma, pancreatic cancer, glioblastoma, glioma, cervical cancer, ovarian cancer, liver cancer (e.g., hepatic carcinoma and hepatoma), bladder cancer, breast cancer, inflammatory breast cancer, Merkel cell carcinoma, colon cancer, colorectal cancer, stomach cancer, urinary bladder cancer, endometrial carcinoma, myeloma (e.g., multiple myeloma), salivary gland, carcinoma, kidney cancer (e.g., renal cell carcinoma and Wilms' tumors), basal cell carcinoma, melanoma, prostate cancer, vulval cancer, thyroid cancer, testicular cancer, esophageal cancer, serous adenocarcinoma or various types of head and neck cancer. In certain embodiments, the cancer treated in accordance with the methods described herein includes desmoplastic melanoma, inflammatory breast cancer, thymoma, rectal cancer, anal cancer, or surgically treatable or non-surgically treatable brain stem glioma. In some instances, the cancer treated in accordance with the methods described herein includes adrenocortical carcinoma, bladder urothelial carcinoma, glioblastoma multiforme, low grade glioma, cervical and endocervical cancers, colon adenocarcinoma, colorectal adenocarcinoma, esophageal carcinoma, head and neck squamous carcinoma, kidney clear cell carcinoma, kidney renal papillary cell carcinoma, liver hepatocellular carcinoma, lung adenocarcinoma, lung adenocarcinoma, lung squamous cell carcinoma, small cell lung carcinoma, ovarian serous adenocarcinoma, pancreas adenocarcinoma, prostate adenocarcinoma, skin cutaneous melanoma, stomach adenocarcinoma, thyroid carcinoma, and/or uterine corpus endometrial carcinoma.

### Methods of Selecting a Treatment for a Subject

Also provided herein are methods of selecting a pharmaceutical composition comprising an immune checkpoint inhibitor for a subject previously identified or diagnosed as having a cancer (e.g., any of the exemplary cancers described herein) and previously identified as having a decreased level of M2 macrophages as compared to a reference level (e.g., any of the exemplary reference levels described herein).

Also provided herein are methods of selecting a pharmaceutical composition comprising an anti-ILT4 binding agent for a subject previously identified or diagnosed as having a cancer (e.g., any of the exemplary cancers described herein) and previously identified as having a an elevated level of M2 macrophages as compared to a reference level (e.g., any of the exemplary reference levels described herein).

Also provided herein are methods of selecting a pharmaceutical composition comprising an anti-CCR8 binding agent for a subject previously identified or diagnosed as having a cancer (e.g., any of the exemplary cancers described herein) and previously identified as having an elevated level of Treg cells and an elevated level of NK cells as compared to reference levels (e.g., any of the exemplary reference levels described herein).

Also provided herein are methods of selecting an anti-CCR8 binding agent and an anti-ILT4 binding agent for a subject previously identified or diagnosed as having a cancer (e.g., any of the exemplary cancers described herein) and previously identified as having an elevated level of Treg cells, an elevated level of NK cells, and an elevated level of NK cells as compared to reference levels (e.g., any of the exemplary reference levels described herein).

### Methods of Selecting a Subject for Treatment

Also provided herein are methods of selecting a subject previously identified or diagnosed as having a cancer (e.g., any of the exemplary cancers described herein) and previously identified as having a decreased level of M2 macrophages as compared to a reference level (e.g., any of the exemplary reference levels described herein) for treatment with a pharmaceutical composition comprising an immune checkpoint inhibitor.

Also provided herein are methods of selecting a subject previously identified or diagnosed as having a cancer (e.g., any of the exemplary cancers described herein) and previously identified as having an elevated level of M2 macrophages as compared to a reference level (e.g., any of the exemplary reference levels described herein) for treatment with a pharmaceutical composition comprising an anti-ILT4 binding agent.

Also provided herein are methods of selecting a subject previously identified or diagnosed as having a cancer (e.g., any of the exemplary cancers described herein) and previously identified as having an elevated level of Treg cells and an elevated level of NK cells as compared to reference levels (e.g., any of the exemplary reference levels described herein) for treatment with a pharmaceutical composition comprising an anti-CCR8 binding agent.

Also provided herein are methods of selecting a subject previously identified or diagnosed as having a cancer (e.g., any of the exemplary cancers described herein) and previously identified as having an elevated level of Treg cells, an elevated level of NK cells, and an elevated level of M2 macrophages as compared to reference levels (e.g., any of the exemplary reference levels described herein) for treatment with an anti-CCR8 binding agent and an anti-ILT4 binding agent.

### EXAMPLES

The invention is further described in the following examples, which do not limit the scope of the invention described in the claims.

### Example 1. Transforming Transcriptomics Data to Immune Cells

We wanted to mine bioinformatics insights from existing databases so that data related to past failures may inform future success in immune-oncology. To this end, we evaluated survival curves to identify patterns. We analyzed cancer-omics data and transformed genomics data into actionable insights. Existing omics data from large and small public and private databases were analyzed and gene expression was translated into immune cell subtypes. Heat-maps were also evaluated to understand response or lack thereof. In particular, a leukocyte gene signature matrix (LM22) with 547 genes was used by the CIBERSORT algorithm to generate gene weights for each cell type. It was then tested on 3 k human transcriptomes. Open source ADAPTS (Automated deconvolution augmentation of profiles for tissue specific cells) software was used for calculating immune-cell enrichment using the LM22 matrix. See, e.g., cran.r-project.org/web/packages/ADAPTS/index.html. Also, ranked genes from LM22 were used in Gene Set Variation Analysis (GSVA). The approach was validated with sorted cells from early-stage non-small cell lung cancer (NSCLC) and follicular lymphoma patients. We were able to increase the efficiency of the analytic approaches and leveraged these insights to prioritize combinations.

### Example 2. Tumor Microenvironment Limits Patient Benefit from PD-1 Inhibitors

Publicly-available data from “The Cancer Genome Atlas” (TCGA) and additional sources was analyzed to identify pattern associated with nonresponse of patients to PD-1 blockade. For example, we analyzed data from Ayers et al., 2017 (J Clin Invest, 127 (8): 2930-2940) that shows progression-free survival (PFS) time versus T cell-inflamed gene expression profile (GEP) score in 244 patients treated with KEYTRUDA, a PD-1 inhibitor. A general pattern of a lack of objective response was observed in patients whose tumors showed low expression levels across the genes, assumed to represent tumors without a T cell-inflamed phenotype. Patients whose tumors had lower T cell-inflamed GEP scores (e.g., less than −0.3) generally showed rapid disease progression, whereas a broad spectrum of progression times was observed for higher T cell-inflamed GEP scores. A large share of patients were identified with additional immune suppressive mechanisms that may interfere with T cell directed tumor killing. Also, poor prognosis in these patients was likely to be driven by unfavorable immune suppressive mechanisms in the tumor microenvironment (TME). Overall, the analysis reveals novel immune suppressive mechanisms in the TME.

### Example 3. Changing Unfavorable TME to Favorable TME for Anti PD-1 Therapeutic Responses

As described in Example 2, our analysis of cancer-omics data revealed novel immune suppressive mechanisms in the TME. Given the role of TME in limiting therapeutic response to PD-1 inhibitors, we aimed to change unfavorable TME to favorable TME for anti PD-1 therapeutic responses. To this end, cancer patients' transcriptomics data was transformed to immune cell types using modified algorithm comprising of 30-45 genes. Next, group of cancer patients (NSCLC/SCLC, etc.) having the addressable dominant MOA were identified. Next, target(s) were identified based on the biology, so as to develop best-in-class drug candidates. This approach would solve major immune suppressive mechanisms that may address “immune escape”. Also, the combination of drugs would address the unfavorable TME, which would result in better outcome for the patients.

### Example 4. High Level of Immune Suppressive Macrophages May be the Reason for Limited PD(L)-1 Response in SCLC Patients

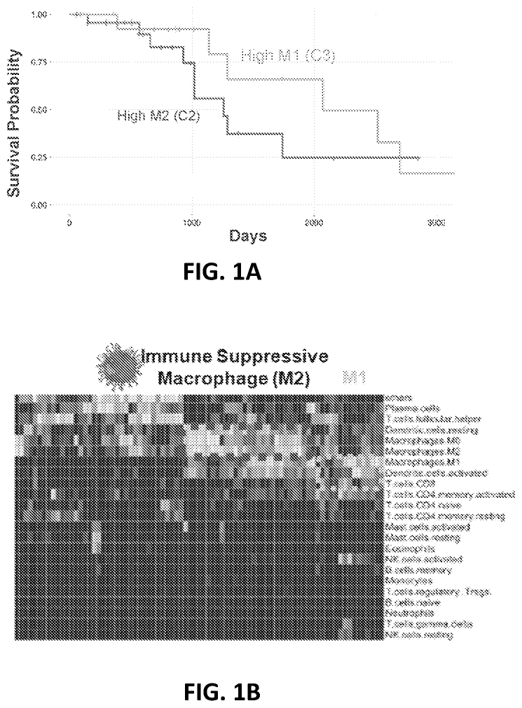

Cancer-omics data of small cell lung cancer (SCLC) patients was re-analyzed to identify pattern associated with limited response of patients to PD(L)-1 therapy. In particular, survival plot and heat map data from George et al., 2015 (Nature, 524(7563): 47-53 (2015)) were reanalyzed using methods described in the foregoing examples. The results are described in FIGS. 1A and 1B. As shown in FIGS. 1A and 1B, patients with enrichment of M1 macrophages (i.e., patients with higher level of M1 macrophages; High M1 (M1 enriched)) showed higher survival probability compared to patients with higher level of immune suppressive M2 macrophages (High M2 (M2 enriched)). Analysis of profiles with elevated levels of resting dendritic cells (DC) and M2 macrophages showed that ˜30% of SCLC patients have high M2 (i.e., immune suppressive macrophage) signatures and poorer prognosis. Thus, our analysis indicated that high level of immune suppressive macrophages within SCLC may be the reason for limited PD(L)-1 response in SCLC patients.

### Example 5. ILT4 Expression is Higher and Immune-T Cells are Lower in SCLC Patients with High M2

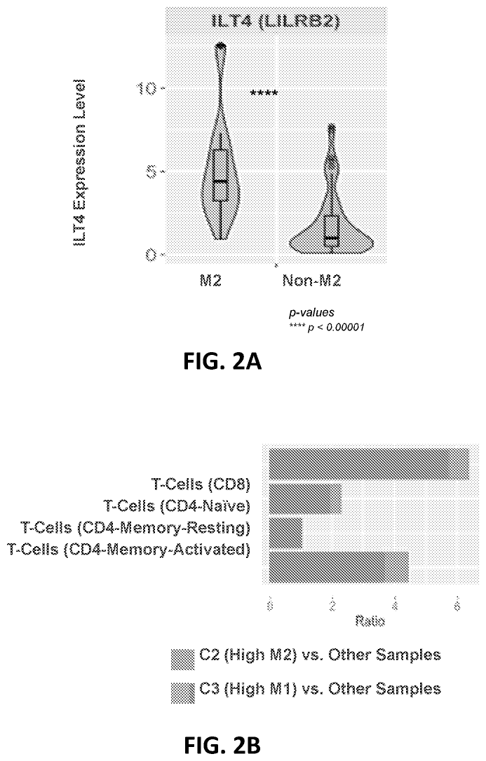

Furthermore, re-analysis of data from SCLC patients showed that ILT4 expression is significantly higher and immune-T cells are lower in SCLC patients that have high M2 (i.e., high level of immune suppressive M2 macrophage). The results are described in FIGS. 2A and 2B. In particular, ILT4 expression was found to be significantly higher in M2 vs. non-M2 samples (FIG. 2A). Also, M2-enriched samples were found to have fewer immune T-cells (FIG. 2B).

### Example 6. NSCLC Patients with High-Level of M2 Macrophages are Likely to be Unresponsive to PD(L)-1 Therapy

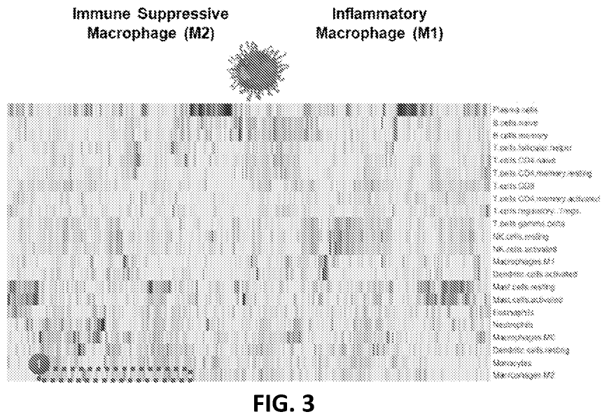

Next, published data of non-small cell lung cancer (NSCLC) patients was re-analyzed to identify pattern associated with limited response of NSCLC patients to PD(L)-1 therapy. In particular, data published by The Cancer Genome Atlas was re-analyzed using methods described in the foregoing examples. The results are described in FIG. 3. As shown in FIG. 3, high expression of immune suppressive M2 macrophages was found in ˜30% patients. Thus, our analysis showed that NSCLC patients with high-level of M2 macrophages are likely to be unresponsiveness to PD(L)-1 therapy.

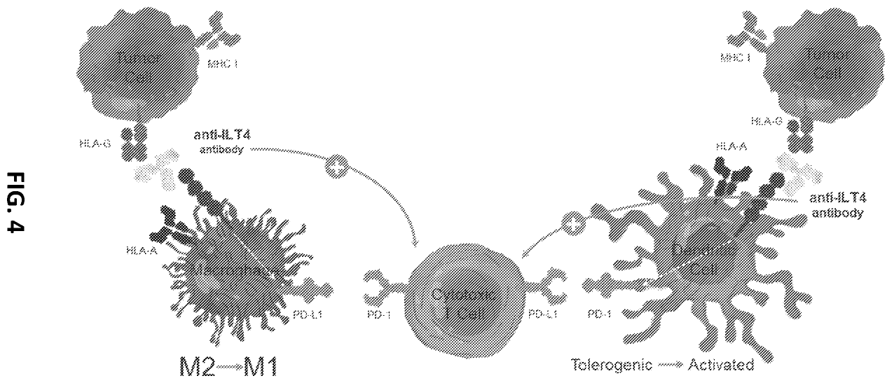

Learnings from the foregoing examples allowed us to rationally develop an anti-ILT4 antibody with an intent of repolarizing M2 (immune suppressive) macrophages to M1 (inflammatory) macrophages. A schematic showing repolarization of M2 (suppressive) macrophages to M1 (inflammatory) macrophages by anti-ILT4 antibody is described in FIG. 4.

### Example 7. NSCLC Patients with Treg Signatures could Benefit from Depleting Anti-CCR8 Antibody Therapy in Combination with Toripalimab

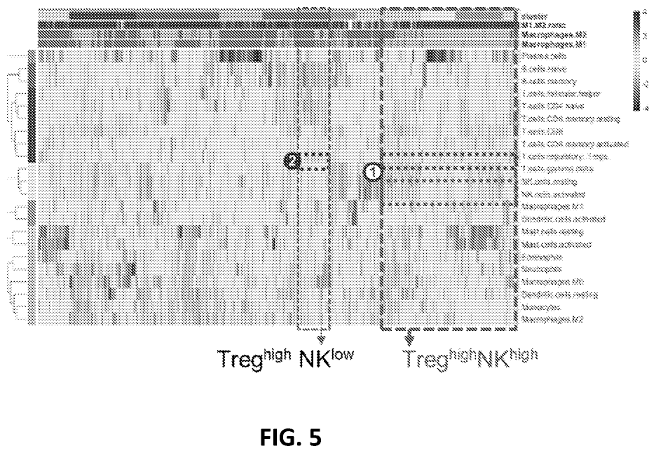

Re-analysis of NSCLC patient data also showed that about 30% of NSCLC patients have high Treg signatures, and these patients could benefit from a combination of depleting anti-CCR8 antibody and toripalimab. The results are described in FIG. 5. As described in FIG. 5, high level of regulatory T-cells (Treg) was found in patients those are not likely to be benefited with PD-1 therapy. Of these, some patients had High Tregs and High NK cells (Population 1; Treg^{high}NK^{high}), while the others had High Tregs and Low NK cells (Population 2; Treg^{high}NK^{low}). The High Treg populations in general appeared to be different from the High M2 groups (described in the foregoing examples). Anti CCR-8 is unlikely to benefit the Treg^{high}NK^{low} patients due to presence of fewer NK cells (i.e., CCRR-8 therapy can benefit these patients, but only when NK cells are present). On the other hand, patients with high levels of Tregs and NK cells (i.e., Treg^{high}NK^{high} patients) are likely to benefit substantially from a toripalimab and anti-CCR8 combination therapy or a similar combination therapy regimen.

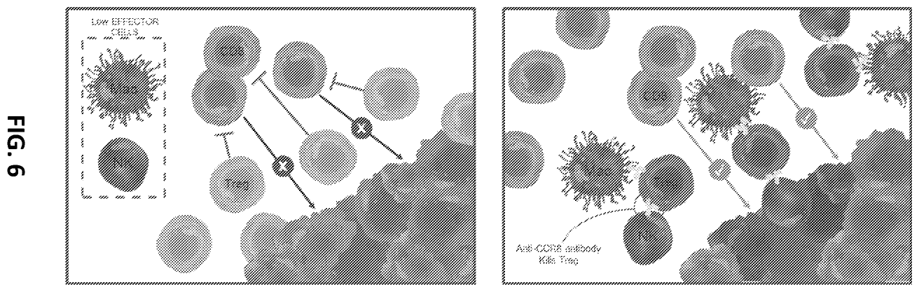

This analysis indicates that depletion of immune suppressive Tregs will allow the cytotoxic T cells to efficiently kill the tumor. In particular, presence of Treg cells may interfere with anti PD(L)1 efficacy, and anti-CCR8 and effector NK cells are needed for an effective Treg depletion. Thus, effective tumor killing can be achieved with CCR8 antibody in the presence of NK cells. A schematic representation of this concept is shown in FIG. 6.

### Example 8. Patient Populations that Will Benefit from Combination Therapies

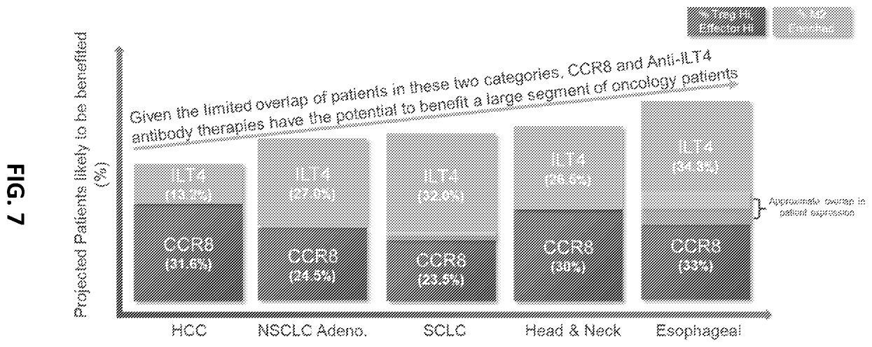

Re-analysis of patient data (as described in the foregoing examples) identified patient populations that are likely to be benefitted from the combination therapies proposed above. In particular, our analysis identified Treg Hi Effector Hi population and M2 Enriched population as the patient populations that will benefit most from therapies comprising ILT4 and CCR8 in combination with toripalimab. The results are described in FIG. 7 and in Table 2 below. The graph in FIG. 7 shows percentage of patients in each of two identified populations (i.e., “Treg Hi, Effector Hi” population and “M2 Enriched” population) that are likely to benefit from the combination therapy. Data is shown for hepatocellular carcinoma (HCC), NSCLC adenocarcinoma, SCLC, head and neck cancers, and esophageal cancer. SCLC M1 and M2 values are based on ADAPTS: LM22 profile. Treg pattern is derived from GSVA using ranked LM22 profile. Given the limited overlap of patients in these two categories, CCR8 and anti-ILT4 antibody therapies might have the potential to benefit a large segment of oncology patients.

TABLE 2
Patient populations likely to benefit from combination therapies

| | Tissue | Dataset, | # | % Treg^{hi} | % M2- |
|---|---|---|---|---|---|
| Tumor | origin | source | Samples | Effector^{hi} | enriched |
| Adrenocortical carcinoma | adrenal | ACC, TCGA | 79 | 31.6 | 38 |
| Bladder urothelial | bladder | BLCA, TCGA | 407 | 31.4 | 15.7 |
| carcinoma | | | | | |
| Glioblastoma multiforme | brain | GBM, TCGA | 164 | 27.6 | 35.5 |
| Low grade glioma | brain | LGG, TCGA | 515 | 23.3 | 32.2 |
| Cervical and endocervical | cervix | CESC, TCGA | 304 | 38.8 | 24 |
| cancers | | | | | |
| Colon adenocarcinoma | colon | COAD, TCGA | 283 | 30 | 20.8 |
| Colorectal | colon | COADREAD, | 376 | 25.2 | 26.9 |
| adenocarcinoma | | TCGA | | | |
| Esophageal carcinoma | esophagus | ESCA, TCGA | 184 | 33 | 34..8 |
| Head and neck squamous | head, neck | HNSC, TCGA | 519 | 30 | 26.5 |
| carcinoma | | | | | |
| Kidney clear cell | kidney | KIRC, TCGA | 533 | 39 | 22.7 |
| carcinoma | | | | | |
| Kidney renal papillary | kidney | KIRP, TCGA | 283 | 33.4 | 30.7 |
| cell carcinoma | | | | | |
| Liver hepatocellular | liver | LIHC, TCGA | 377 | 31.6 | 13.2 |
| carcinoma | | | | | |
| Lung adenocarcinoma | lung | LUAD, TCGA | 496 | 27 | 25 |
| Lung adenocarcinoma | lung | LUAD, | 169 | 22 | 29 |
| | | Singapore | | | |
| Lung squamous cell | lung | TCGA LUSC | 491 | 35 | 38.4 |
| carcinoma | | | | | |
| Small cell lung | lung | SCLC, | 81 | 28 | 33.3 |
| carcinoma | | Cologne | | | |
| Ovarian serous | ovary | OV, TCGA | 300 | 41 | 18.3 |
| adenocarcinoma | | | | | |
| Pancreas adenocarcinoma | pancreas | PAAD, TCGA | 178 | 24.7 | 32 |
| Prostate adenocarcinoma | prostate | PRAD, TCGA | 497 | 28.2 | 18.3 |
| Skin cutaneous | skin | SKCM, TCGA | 358 | 33.8 | 17 |
| melanoma | | | | | |
| Stomach adenocarcinoma | stomach | STAD, TCGA | 403 | 31.5 | 25.8 |
| Thyroid carcinoma | thyroid | THCA, TCGA | 501 | 22.8 | 30.3 |
| Uterine corpus | uterus | UCEC, TCGA | 175 | 32.6 | 32 |
| endometrial carcinoma | | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a cancer, the method comprising:
administering a therapeutically effective amount of an immune checkpoint inhibitor to a subject previously identified or diagnosed as having a cancer and previously identified as having a decreased level of M2 macrophages as compared to a reference level.

2. A method of selecting a pharmaceutical composition comprising an immune checkpoint inhibitor for a subject previously identified or diagnosed as having a cancer and previously identified as having a decreased level of M2 macrophages as compared to a reference level.

3. A method of selecting a subject previously identified or diagnosed as having a cancer and previously identified as having a decreased level of M2 macrophages as compared to a reference level for treatment with a pharmaceutical composition comprising an immune checkpoint inhibitor.

4. A method of predicting efficacy of treatment with a pharmaceutical composition comprising an immune checkpoint inhibitor in a subject in need thereof, the method comprising:
determining that a subject diagnosed or identified as having a cancer and identified as having a decreased level of M2 macrophages as compared to a reference level has an increased likelihood of having a positive therapeutic response to treatment with the pharmaceutical composition; or
determining that a subject diagnosed or identified as having a cancer and identified as having an elevated level of M2 macrophages as compared to a reference level has a decreased likelihood of having a positive therapeutic response to treatment with the pharmaceutical composition.

5. The method of any one of claims 1-4, wherein the immune checkpoint inhibitor is an anti-PD-1 antibody or an antigen-binding fragment thereof.

6. The method of any one of claims 1-4, wherein the immune checkpoint inhibitor is an anti-PD-L1 antibody or an antigen-binding fragment thereof.

7. The method of any one of claims 1-6, wherein the method further comprises:
determining a level of M2 macrophages in a sample obtained from the subject; and
identifying the subject as having a decreased level of M2 macrophages as compared to the reference level.

8. A method of treating a subject having a cancer, the method comprising:
administering a therapeutically effective amount of an anti-ILT4 binding agent to a subject previously identified or diagnosed as having a cancer and previously identified as having an elevated level of M2 macrophages as compared to a reference level.

9. A method of selecting a pharmaceutical composition comprising an anti-ILT4 binding agent for a subject previously identified or diagnosed as having a cancer and previously identified as having a an elevated level of M2 macrophages as compared to a reference level.

10. A method of selecting a subject previously identified or diagnosed as having a cancer and previously identified as having an elevated level of M2 macrophages as compared to a reference level for treatment with a pharmaceutical composition comprising an anti-ILT4 binding agent.

11. The method of any one of claims 8-10, wherein the anti-ILT4 binding agent is an anti-ILT4 antibody or an antigen-binding fragment thereof.

12. The method of any one of claims 8-11, wherein the method further comprises administering an immune checkpoint inhibitor to the subject.

13. The method of claim 12, wherein the immune checkpoint inhibitor is an anti-PD-1 antibody or an antigen-binding fragment thereof.

14. The method of claim 12, wherein the immune checkpoint inhibitor is an anti-PD-L1 antibody or an antigen-binding fragment thereof.

15. The method of any one of claims 8-14, wherein the method further comprises:
determining a level of M2 macrophages in a sample obtained from the subject; and
identifying the subject as having an elevated level of M2 macrophages as compared to the reference level.

16. The method of any one of claims 1-15, wherein the M2 macrophages have an elevated level of ILT4 expression as compared to a reference level.

17. The method of any one of claims 1-16, wherein the reference level of M2 macrophages is a level of M2 macrophages in a population of healthy subjects.

18. The method of any one of claims 1-16, wherein the reference level of M2 macrophages is a 75% percentile of the median level of the number of M2 macrophages in a healthy patient population.

19. The method of any one of claims 1-16, wherein the reference level of M2 macrophages is a 80% percentile of the median level of M2 macrophages in a healthy patient population.

20. The method of any one of claims 1-16, wherein the reference level of M2 macrophages is a 85% percentile of the median level of M2 macrophages in a healthy patient population.

21. The method of any one of claims 1-16, wherein the reference level of M2 macrophages is a 90% percentile of the median level of M2 macrophages in a healthy patient population.

22. The method of any one of claims 1-16, wherein the reference level of M2 macrophages is a 95% percentile of the median level of M2 macrophages in a healthy patient population.

23. The method of any one of claims 1-22, wherein the cancer is a solid tumor.

24. The method of claim 23, wherein the solid tumor is non-small cell lung cancer, hepatocellular carcinoma, small-cell lung cancer, head and neck cancer, or esophageal cancer.

25. The method of any one of claims 1-22, wherein the cancer is a hematological cancer.

26. A method of treating a subject having a cancer, the method comprising:
administering a therapeutically effective amount of an anti-CCR8 binding agent to a subject previously identified or diagnosed as having a cancer and previously identified as having an elevated level of Treg cells and an elevated level of NK cells as compared to reference levels.

27. A method of selecting a pharmaceutical composition comprising an anti-CCR8 binding agent for a subject previously identified or diagnosed as having a cancer and previously identified as having an elevated level of Treg cells and an elevated level of NK cells as compared to reference levels.

28. A method of selecting a subject previously identified or diagnosed as having a cancer and previously identified as having an elevated level of Treg cells and an elevated level of NK cells as compared to reference levels for treatment with a pharmaceutical composition comprising an anti-CCR8 binding agent.

29. A method of predicting efficacy of treatment with a pharmaceutical composition comprising an anti-CCR8 binding agent in a subject in need thereof, the method comprising:
determining that a subject diagnosed or identified as having a cancer and identified as having an elevated level of Treg cells and an elevated level of NK cells as compared to reference levels has an increased likelihood of having a positive therapeutic response to treatment with the pharmaceutical composition; or
determining that a subject diagnosed or identified as having a cancer and identified an elevated level of Treg cells and a decreased level of NK cells as compared to reference levels has a decreased likelihood of having a positive therapeutic response to treatment with the pharmaceutical composition.

30. The method of any one of claims 26-29, wherein the anti-CCR8 binding agent is an anti-CCR8 antibody or antigen-binding fragment thereof.

31. The method of any one of claims 26-30, wherein the method further comprises administering an immune checkpoint inhibitor to the subject.

32. The method of claim 31, wherein the immune checkpoint inhibitor is an anti-PD-1 antibody or an antigen-binding fragment thereof.

33. The method of claim 31, wherein the immune checkpoint inhibitor is an anti-PD-L1 antibody or an antigen-binding fragment thereof.

34. The method of any one of claims 26-33, wherein the method further comprises:
determining a level of Treg cells and NK cells in a sample obtained from the subject; and
identifying the subject as having an elevated level of T reg cells and NK cells as compared to the reference levels.

35. The method of any one of claims 26-34, wherein the reference level of Treg cells is a level of Treg cells in a population of healthy subjects.

36. The method of any one of claims 26-34, wherein the reference level of Treg cells is a 75% percentile of the median level of the number of Treg cells in a healthy patient population.

37. The method of any one of claims 26-34, wherein the reference level of Treg cells is a 80% percentile of the median level of Treg cells in a healthy patient population.

38. The method of any one of claims 26-34, wherein the reference level of Treg cells is a 85% percentile of the median level of Treg cells in a healthy patient population.

39. The method of any one of claims 26-34, wherein the reference level of Treg cells is a 90% percentile of the median level of Treg cells in a healthy patient population.

40. The method of any one of claims 26-34, wherein the reference level of Treg cells is a 95% percentile of the median level of Treg cells in a healthy patient population.

41. The method of any one of claims 26-40, wherein the reference level of NK cells is a level of NK cells in a population of healthy subjects.

42. The method of any one of claims 26-40, wherein the reference level of NK cells is a 75% percentile of the median level of the number of NK cells in a healthy patient population.

43. The method of any one of claims 26-40, wherein the reference level of NK cells is a 80% percentile of the median level of NK cells in a healthy patient population.

44. The method of any one of claims 26-40, wherein the reference level of NK cells is a 85% percentile of the median level of NK cells in a healthy patient population.

45. The method of any one of claims 26-40, wherein the reference level of NK cells is a 90% percentile of the median level of NK cells in a healthy patient population.

46. The method of any one of claims 26-40, wherein the reference level of NK cells is a 95% percentile of the median level of NK cells in a healthy patient population.

47. The method of any one of claims 26-46, wherein the cancer is a solid tumor.

48. The method of claim 47, wherein the solid tumor is non-small cell lung cancer, hepatocellular carcinoma, small-cell lung cancer, head and neck cancer, or esophageal cancer.

49. The method of any one of claims 26-46, wherein the cancer is a hematological cancer.

50. A method of treating a subject having a cancer, the method comprising:
administering a therapeutically effective amount of an anti-CCR8 binding agent and a therapeutically effective amount of anti-ILT4 binding agent to a subject previously identified or diagnosed as having a cancer and previously identified as having an elevated level of Treg cells, an elevated level of NK cells, and an elevated number of M2 macrophages as compared to reference levels.

51. A method of selecting an anti-CCR8 binding agent and an anti-ILT4 binding agent for a subject previously identified or diagnosed as having a cancer and previously identified as having an elevated level of Treg cells, an elevated level of NK cells, and an elevated level of NK cells as compared to reference levels.

52. A method of selecting a subject previously identified or diagnosed as having a cancer and previously identified as having an elevated level of Treg cells, an elevated level of NK cells, and an elevated level of M2 macrophages as compared to reference levels for treatment with an anti-CCR8 binding agent and an anti-ILT4 binding agent.

53. A method of predicting efficacy of treatment with an anti-CCR8 binding agent and an anti-ILT4 binding agent in a subject in need thereof, the method comprising:
determining that a subject diagnosed or identified as having a cancer and identified as having an elevated level of Treg cells, an elevated level of NK cells, and an elevated level of M2 macrophages as compared to reference levels has an increased likelihood of having a positive therapeutic response to treatment with the pharmaceutical composition; or
determining that a subject diagnosed or identified as having a cancer and not identified as having an elevated level of Treg cells, an elevated level of NK cells, and an elevated level of M2 macrophages as compared to reference levels has a decreased likelihood of having a positive therapeutic response to treatment with an anti-CCR8 binding agent and an anti-ITL4 binding agent.

54. The method of any one of claims 50-53, wherein the anti-CCR8 binding agent is an anti-CCR8 antibody or antigen-binding fragment thereof.

55. The method of any one of claims 50-54, wherein the anti-ILT4 binding agent is an anti-ILT4 antibody or an antigen-binding fragment thereof.

56. The method of any one of claims 50-55, wherein the method further comprises administering an immune checkpoint inhibitor to the subject.

57. The method of claim 56, wherein the immune checkpoint inhibitor is an anti-PD-1 antibody or an antigen-binding fragment thereof.

58. The method of claim 56, wherein the immune checkpoint inhibitor is an anti-PD-L1 antibody or an antigen-binding fragment thereof.

59. The method of any one of claims 50-58, wherein the method further comprises:
determining a level of Treg cells, NK cells, and M2 macrophages in a sample obtained from the subject; and
identifying the subject as having an elevated level of T reg cells, an elevated level of NK cells, and an elevated level of M2 macrophages as compared to the reference levels.

60. The method of any one of claims 50-59, wherein the reference level of Treg cells is a level of Treg cells in a population of healthy subjects.

61. The method of any one of claims 50-59, wherein the reference level of Treg cells is a 75% percentile of the median level of the number of Treg cells in a healthy patient population.

62. The method of any one of claims 50-59, wherein the reference level of Treg cells is a 80% percentile of the median level of Treg cells in a healthy patient population.

63. The method of any one of claims 50-59, wherein the reference level of Treg cells is a 85% percentile of the median level of Treg cells in a healthy patient population.

64. The method of any one of claims 50-59, wherein the reference level of Treg cells is a 90% percentile of the median level of Treg cells in a healthy patient population.

65. The method of any one of claims 50-59, wherein the reference level of Treg cells is a 95% percentile of the median level of Treg cells in a healthy patient population.

66. The method of any one of claims 50-65, wherein the reference level of NK cells is a level of NK cells in a population of healthy subjects.

67. The method of any one of claims 50-65, wherein the reference level of NK cells is a 75% percentile of the median level of the number of NK cells in a healthy patient population.

68. The method of any one of claims 50-65, wherein the reference level of NK cells is a 80% percentile of the median level of NK cells in a healthy patient population.

69. The method of any one of claims 50-65, wherein the reference level of NK cells is a 85% percentile of the median level of NK cells in a healthy patient population.

70. The method of any one of claims 50-65, wherein the reference level of NK cells is a 90% percentile of the median level of NK cells in a healthy patient population.

71. The method of any one of claims 50-65, wherein the reference level of NK cells is a 95% percentile of the median level of NK cells in a healthy patient population.

72. The method of any one of claims 50-71, wherein the reference level of M2 macrophages is a level of M2 macrophages in a population of healthy subjects.

73. The method of any one of claims 50-71, wherein the reference level of M2 macrophages is a 75% percentile of the median level of the number of M2 macrophages in a healthy patient population.

74. The method of any one of claims 50-71, wherein the reference level of M2 macrophages is a 80% percentile of the median level of M2 macrophages in a healthy patient population.

75. The method of any one of claims 50-71, wherein the reference level of M2 macrophages is a 85% percentile of the median level of M2 macrophages in a healthy patient population.

76. The method of any one of claims 50-71, wherein the reference level of M2 macrophages is a 90% percentile of the median level of M2 macrophages in a healthy patient population.

77. The method of any one of claims 50-71, wherein the reference level of M2 macrophages is a 95% percentile of the median level of M2 macrophages in a healthy patient population.

78. The method of any one of claims 50-77, wherein the cancer is a solid tumor.

79. The method of claim 78, wherein the solid tumor is non-small cell lung cancer, hepatocellular carcinoma, small-cell lung cancer, head and neck cancer, or esophageal cancer.

80. The method of any one of claims 50-77, wherein the cancer is a hematological cancer.
